(19) United States
(12) Patent Application Publication
(10) Pub. No.: US 2026/0232244 A1
(43) Pub. Date: Aug. 13, 2026

(54) BIOLOGICAL SENSOR

(71) Applicant: NITTO DENKO CORPORATION, Ibaraki-shi, Osaka (JP)

(72) Inventor: Sota KONDO, Ibaraki-shi, Osaka (JP)

(21) Appl. No.: 19/152,529

(22) PCT Filed: Feb. 1, 2024
(86) PCT No.: PCT/JP2024/003371

§ 371 Date: Jul. 31, 2025

(30) Foreign Application Priority Data
Feb. 7, 2023 (JP) 2023-017080

(51) Int. Cl. A61B 5/257; A61B 5/00

(52) U.S. Cl. CPC A61B 5/257; A61B 5/6833

(57) ABSTRACT

A biological sensor to be attached to a living body includes: a sensor body configured to obtain biological information; a first layer member having a projecting shape so as to cover the sensor body; a second layer member that is attached to a surface of the first layer member and includes an attachment surface to be attached to the living body, the attachment surface being opposite to a direction in which the first layer member projects; and a housing space for the sensor body, the housing space being enclosed by the first layer member and the second layer member, in which a permeating amount of water vapor entering the housing space through the second layer member at a first temperature is smaller than a permeating amount of water vapor exiting from the housing space through the first layer member at a second temperature lower than the first temperature.

### TECHNICAL FIELD

The present invention relates to a biological sensor.

### BACKGROUND ART

A biological sensor configured to perform measurement of biological information, such as an electrocardiogram waveform, a pulse wave, an electroencephalogram, an electromyogram, or the like, is used in medical institutions, such as a hospital, a clinic, and the like, nursing facilities, ones' homes, and the like. The biological sensor includes a biological electrode configured to obtain biological information of subjects by contact with their living body. When measuring such biological information, the biological sensor is attached to skin of a subject, and an electric signal of the biological information is obtained by the biological electrode. As a result, measurement of the biological information is performed.

As such a biological sensor, for example, a biological sensor including a sensor body, an electrode, a first layer member, and a second layer member is disclosed. In this biological sensor, the first layer member is formed by stacking a cover on an upper sheet so as to have a space configured to house the sensor body, and the second layer member is attached to a surface of the first layer member on the living body side and is formed such that the sensor body is disposed and the electrode is exposed (see, for example, PTL 1).

This biological sensor includes a first adhesive layer provided on a surface of the first layer member facing the living body and a second adhesive layer provided on a surface of the second layer member facing the living body, and obtains biological information by the electrode that is attached to the first adhesive layer and exposed from the second layer member in a state of attaching the first adhesive layer and the second adhesive layer to skin.

### CITATION LIST

### Patent Literature

PTL 1: Japanese Patent No. 6,947,955

### SUMMARY OF THE INVENTION

### Technical Problem

Herein, when the biological sensor in PTL 1 is attached to the skin of a subject, water vapor derived from sweat generated from the skin can possibly pass through the space inside the biological sensor and adhere to the sensor body or the like disposed inside the biosensor. In order for the biological sensor to stably measure an electric signal indicating biological information on the subject, it is important to prevent water vapor, which passes through the space inside the biological sensor, from adhering to the sensor body or the like and causing dew condensation.

In one aspect of the present invention, it is an object to provide a biological sensor capable of reducing the occurrence of dew condensation on a member disposed in a space inside the biological sensor.

### SOLUTION TO THE PROBLEM

According to one aspect of the present invention, a biological sensor to be attached to a living body includes:
a sensor body configured to obtain biological information;
a first layer member having a projecting shape so as to cover the sensor body;
a second layer member that is attached to a surface of the first layer member and includes an attachment surface to be attached to the living body, the attachment surface being opposite to a direction in which the first layer member projects; and
a housing space for the sensor body, the housing space being enclosed by the first layer member and the second layer member, in which
a permeating amount of water vapor entering the housing space through the second layer member at a first temperature is smaller than a permeating amount of water vapor exiting from the housing space through the first layer member at a second temperature lower than the first temperature.

### Advantageous Effects of the Invention

According to one aspect of the biological sensor of the present invention, it is possible to reduce the occurrence of dew condensation on a member disposed in a space inside the biological sensor.

### BRIEF DESCRIPTION OF THE DRAWINGS

FIG. 1 is a perspective view illustrating an entire configuration of a biological sensor according to an embodiment of the present invention.

FIG. 2 is a plan view illustrating examples of parts of the biological sensor.

FIG. 3 is a longitudinal cross-sectional view of the biological sensor taken along the line I-I in FIG. 1.

FIG. 4 is an explanatory view illustrating the biological sensor of FIG. 1 attached to the chest of a subject.

### DESCRIPTION OF THE EMBODIMENTS

In the following, embodiments of the present invention will be described in detail. For ease of understanding to the description, the same components in the drawings are denoted by the same symbols, and duplicate description is omitted. Also, the scale of the members in the drawings may differ from the actual scale. In this specification, the expression indicating a numerical range: “from . . . through . . . ” means that the numerical value described after “from” and the numerical value described after “through” are included in that numerical range as a lower limit and an upper limit, unless otherwise specified.

### Biological Sensor

A biological sensor according to the present embodiment will be described. The living body refers to, for example, a human body (human) and animals, such as cattle, horses, pigs, chickens, dogs, cats, and the like. The biological sensor according to the present embodiment is suitably used for the living body, especially for a human body. The present embodiment will be described taking, as an example, a case in which the living body is of a human.

The biological sensor according to the present embodiment is an attachment-type biological sensor configured to be attached to a part of a living body (e.g., skin, scalp, forehead, or the like), thereby performing measurement of biological information. In the present embodiment, a description will be given of a case in which the biological sensor is attached to the skin of a human and measures an electric signal (biological signal) indicating biological information on the human.

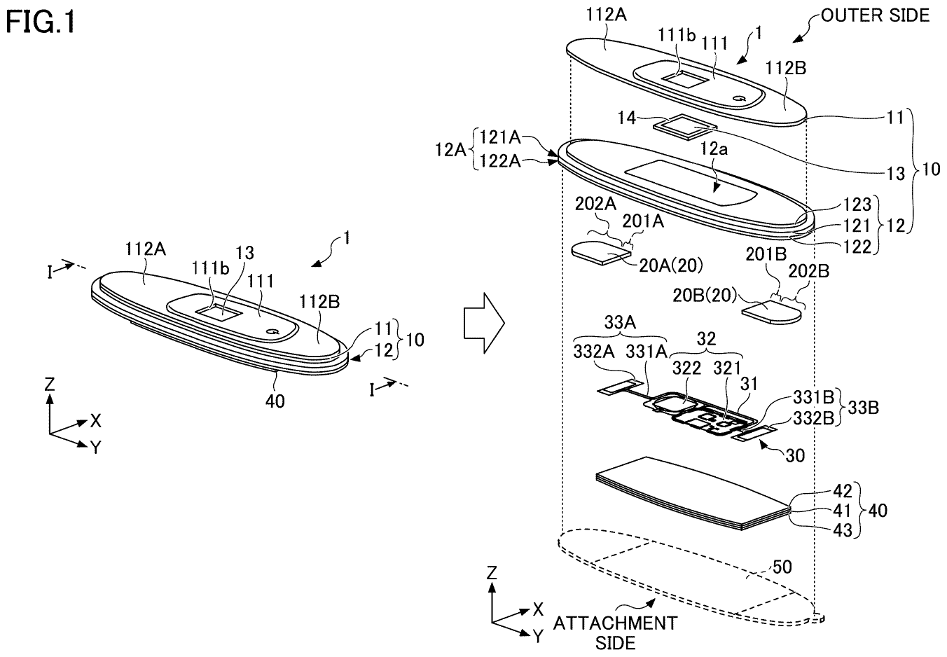

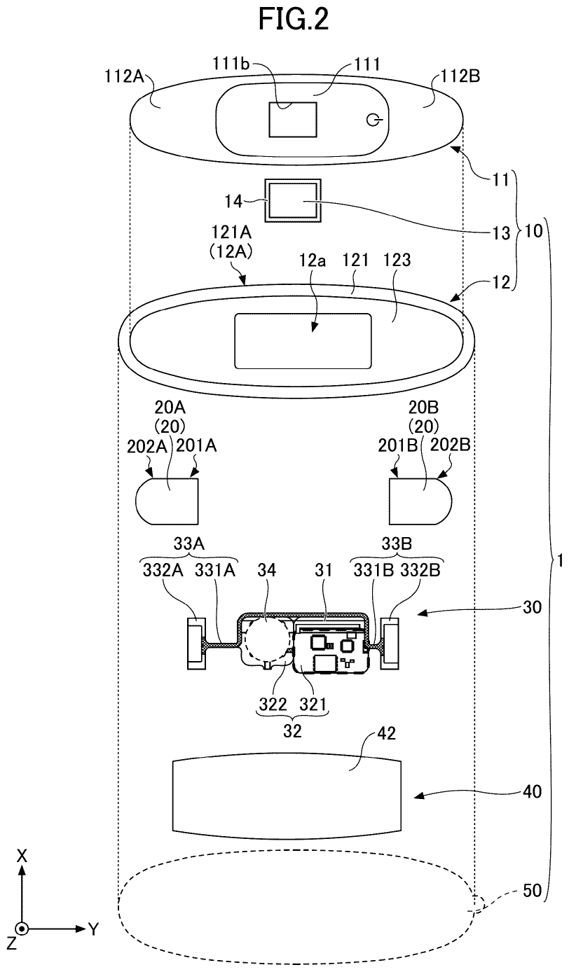

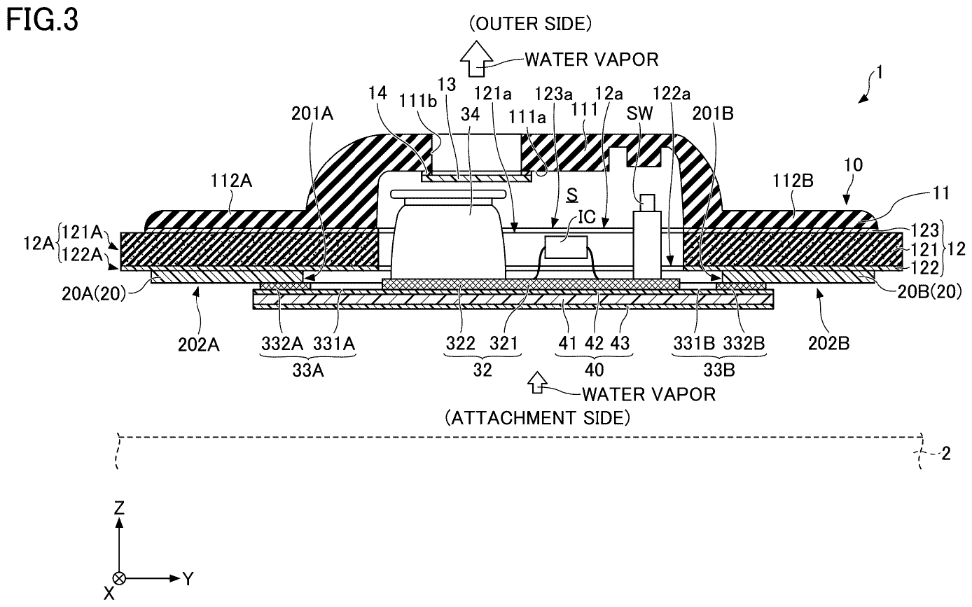

FIG. 1 is a perspective view illustrating the entire configuration of the biological sensor according to the present embodiment. The left-hand view of FIG. 1 illustrates the external appearance of the biological sensor according to the present embodiment, and the right-hand view of FIG. 1 illustrates a state in which the parts of the biological sensor according to the present embodiment are exploded. FIG. 2 is a plan view illustrating examples of the parts of the biological sensor. FIG. 3 is a longitudinal cross-sectional view of the biological sensor taken along the line I-I in FIG. 1.

As illustrated in FIGS. 1 and 2, a biological sensor 1 is a plate-like (sheet-like) member formed in a substantially elliptical shape in a plan view. As illustrated in FIGS. 2 and 3, the biological sensor 1 includes a first layer member 10, an electrode 20, a sensor portion 30, and a second layer member 40, and is formed by stacking the first layer member 10, the electrode 20, and the second layer member 40 in this order from the first layer member 10 side toward the second layer member 40 side. A housing space S, which is enclosed by the first layer member 10, the electrode 20, and the second layer member 40, is formed inside the biological sensor 1. According to the biological sensor 1, the first layer member 10, the electrode 20, and the second layer member 40 form an attachment surface to a skin 2, which is the living body. The biological sensor 1 attaches the attachment surface to the skin 2 and measures a potential difference (polarization voltage) between the skin 2 and the electrode 20, thereby measuring an electric signal (biological signal) indicating biological information on a subject.

In FIGS. 1 to 3, using a three-dimensional orthogonal coordinate system having three axis directions (X-axis direction, Y-axis direction, and Z-axis direction), the transverse direction of the biological sensor 1 is an X-axis direction, the longitudinal direction of the biological sensor is a Y-axis direction, and the height direction (thickness direction) of the biological sensor is a Z-axis direction. The side (outer side) opposite to the side on which the biological sensor 1 is attached to the living body (subject) (attachment side) is referred to as a +Z-axis direction, and the attachment side is referred to as a −Z-axis direction. In the following description, for the sake of convenience, the +Z-axis direction may be referred to as an upper side or above, and the −Z-axis direction may be referred to as a lower side or below. However, this does not represent a universal vertical relationship.

The biological signal is, for example, an electric signal indicating an electrocardiogram waveform, an electroencephalogram, a pulse, or the like.

Moreover, the biological sensor 1 is not required to include the electrode 20 as long as the sensor portion 30 can measure the biological signal by infrared rays or the like. In this case, the housing space S may be formed by being enclosed by the first layer member 10 and the second layer member 40.

According to the biological sensor 1, a permeating amount of water vapor at a first temperature T1 (hereinafter, referred to as a first permeating amount M1) is made smaller than a permeating amount of water vapor at a second temperature T2 (hereinafter, referred to as a second permeating amount M2). The first permeating amount M1 is of the water vapor entering the housing space S through the second layer member 40, a member forming at least a part of the attachment surface among members constituting the biological sensor 1. The second permeating amount M2 is of the water vapor exiting from the housing space S through the first layer member 10. This enables the biological sensor 1 to allow the water vapor derived from sweat on the surface of the skin 2 to pass through the housing space S inside the biological sensor 1 and to be easily released to the exterior, thereby suppressing the occurrence of dew condensation in the sensor portion 30 disposed inside the biological sensor 1.

The first temperature T1 is a temperature within the same or substantially the same range as the skin 2 and is, for example, from 33° C. through 38° C., preferably from 35° C. through 37° C., and more preferably around 36° C.

The second temperature T2 is a temperature lower than the first temperature T1 and is, for example, from 27° C. through 32° C., preferably from 29° C. through 31° C., and more preferably around 30° C.

Since the first layer member 10, the electrode 20, and the second layer member 40 in the biological sensor 1 form the attachment surface to the skin 2, the first layer member 10, the electrode 20, and the second layer member 40 are the members forming the attachment surface. Among these members, the member having the largest amount of the water vapor derived from sweat generated from the skin 2 is the second layer member 40, which is located closest to the skin 2 and at which the sensor portion 30 is provided. The permeating amount of the water vapor entering the housing space S from the skin 2 substantially corresponds to the permeating amount of the second layer member 40. Therefore, the first permeating amount M1 is set as the permeating amount of the second layer member 40 at the first temperature.

In the biological sensor 1, the first layer member 10 is provided with a moisture permeable film 13, and the moisture permeable film 13 has a higher moisture permeability than the first layer member 10, as described below. Since the permeating amount of the water vapor exiting from the housing space S to the exterior through the first layer member 10 substantially corresponds to the permeating amount of the moisture permeable film 13, the second permeating amount M2 is set as the permeating amount of the moisture permeable film 13 at the second temperature.

The first permeating amount M1 is the permeating amount of the water vapor entering the housing space S through the second layer member 40, a member which forms at least a part of the attachment surface as described above. Therefore, the first permeating amount M1 can be said to be the permeating amount of the second layer member 40 at the first temperature T1.

The second permeating amount M2 is the permeating amount of the water vapor exiting from the housing space S through the first layer member 10 at the second temperature T2 as described above, and is influenced by the permeating amount of the moisture permeable film 13 of the first layer member 10, which has a high moisture permeability. Therefore, the second permeating amount M2 can be said to be the permeating amount of the moisture permeable film 13 at the second temperature T2.

The permeating amount can be obtained from the following formula (1):

$$Permeating Amount [ g / day ] = Moisture Permeability [ g / ( {m}^{2} \cdot day ) ] \times Surface Area [ {m}^{2} ] \qquad ( 1 )$$

That is, the first permeating amount M1 is obtained from the following formula (1-1):

$$( 1 - 1 )$$

$$First permeating Amount M 1 [ g / day ] = Moisture Permeability of Second Layer Member 40 [ g / ( {m}^{2} \cdot day ) ] \times Surface Area of Second Layer Member 40 in Bottom View [ {m}^{2} ]$$

The second permeating amount M2 is obtained from the following formula (1-2):

$$Second Perneating Amount M 2 [ g / day ] = Moisture Permeability of Moisture Permeable Film 13 [ g / {m}^{2} \cdot day ) ] \times Surface Area of Through - Hole 111 b Provided in Projection 111 of Cover Member 11 [ {m}^{2} ] \qquad ( 1 - 2 )$$

Note that the surface area of a through-hole 111b provided in a projection 111 of a cover member 11 means the surface area of the moisture permeable film 13 exposed from the through-hole 111b.

### First Layer Member

As illustrated in FIGS. 1 and 2, the first layer member 10 includes the cover member 11, an upper sheet 12, and the moisture permeable film 13. The first layer member 10 has a space inside so as to cover a sensor body 32 included in the sensor portion 30, and has a shape that projects in a substantially dome shape toward the height direction (+Z axis direction) in FIG. 1. The upper sheet 12 is slightly larger than the cover member 11 in a plan view.

### Cover Member

As illustrated in FIG. 3, the cover member 11 is positioned on the outermost side (+Z-axis direction) of the biological sensor 1, and is adhered to the upper surface of the upper sheet 12. The cover member 11 includes: the projection 111 that projects in a substantially dome shape in the height direction (+Z-axis direction) in FIG. 1, the projection 111 being in a center region in the longitudinal direction (Y-axis direction); and flat portions 112A and 112B provided at both ends of the cover member 11 in the longitudinal direction (Y-axis direction). The upper and lower surfaces of the projection 111, and the upper and lower surfaces of the flat portions 112A and 112B may be formed to be flat.

The cover member 11 has an opening on the inner side (attachment side) of the projection 111 so as to have a recess 111a formed in a recessed shape on the skin 2 side. The recess 111a only needs to have at least a part of the housing space S and have a size sufficient to house at least a part of the sensor portion 30. The housing space S in which the sensor portion 30 is housed is formed, on the inner side (attachment side) of the projection 111, by the recess 11la at the inner surface of the projection 111, the electrode 20, and the second layer member 40.

The cover member 11 may have the through-hole 111b in the recess 111a, which communicates with the housing space S. The size of the through-hole 111b is not particularly limited and may be any appropriate size. Moreover, the shape of the through-hole 111b is not particularly limited and may be, for example, circular, rectangular, elliptical, or the like.

Typically, the cover member 11 may be formed using a flexible material, such as crosslinked rubber or the like. Examples of the crosslinked rubber include silicone rubber, fluororubber, urethane rubber, natural rubber, acrylic rubber, butadiene rubber, isoprene rubber, styrene-butadiene copolymer rubber, nitrile rubber, hydrogenated nitrile rubber, chloroprene rubber, ethylene-propylene copolymer rubber, chlorinated polyethylene rubber, chlorosulfonated polyethylene rubber, butyl rubber, halogenated butyl rubber, and the like. The cover member 11 may be formed by stacking the flexible material on the surface of a support that is formed of a base resin, such as polyethylene terephthalate (PET) or the like. The cover member 11 formed using the flexible material or the like protects the sensor portion 30 disposed in the housing space S of the cover member 11, and absorbs an impact applied to the biological sensor 1 from the upper surface side to reduce the impact applied to the sensor portion 30.

The thickness of the upper surface and the side walls of the projection 111 may be larger than that of the flat portions 112A and 112B. Thus, the flexibility of the projection 111 can be lower than that of the flat portions 112A and 112B, and the sensor portion 30 can be protected from an external force applied to the biological sensor 1.

The thickness of the upper surface and the side walls of the projection 111 can be appropriately designed and may be, for example, from 1.5 mm through 3 mm. The thickness of the flat portions 112A and 112B can also be appropriately designed and may be, for example, from 0.5 mm through 1 mm.

The flat portions 112A and 112B, which are thinner, have higher flexibility than that of the projection 111. Thus, when the biological sensor 1 is attached to the skin 2, they readily deform in accordance with deformation of the surface of the skin 2 caused by body movements, such as extension, bending, twisting, and the like. This can reduce stress applied to the flat portions 112A and 112B in response to deformation of the surface of the skin 2, and can suppress peeling of the biological sensor 1 off from the skin 2.

The outer peripheral portions of the flat portions 112A and 112B may have a shape in which the thickness gradually decreases toward the respective ends. This can further increase the flexibility of the outer peripheral portions of the flat portions 112A and 112B, and can improve sensation during attachment of the biological sensor 1 to the skin 2 compared to a case in which the thickness of the outer peripheral portions of the flat portions 112A and 112B are not made smaller. As described below, the upper sheet 12 can reduce the stress applied to the flat portions 112A and 112B upon deformation of the surface of the skin 2.

The moisture permeability of the cover member 11 is 350 g/(m²·day) or less, preferably 330 g/(m²·day) or less, and more preferably 310 g/(m²·day) or less. As long as the moisture permeability of the cover member 11 is 350 g/(m²·day) or less, when water vapor derived from sweat or the like generated from the skin 2, to which the biological sensor 1 is attached, reaches the cover member 11, the water vapor can be released to the exterior of the biological sensor 1 through the cover member 11.

No particular limitation is imposed on the calculation method of the moisture permeability of the cover member 11, and a typical method can be used. For example, the moisture permeability of the cover member 11 may be calculated in accordance with the following procedure.
(1) A weighing bottle having an opening with a predetermined area S is provided, and a sufficient amount of water is charged into the weighing bottle such that the liquid surface is positioned below the opening.
(2) A part or all of the cover member 11 is disposed as a measurement sample over the entirety of the opening of the weighing bottle such that no tension is generated in the cover member 11, and the measurement sample is fixed to the weighing bottle, thereby sealing the weighing bottle.
(3) A total mass M1 of the measurement sample, the water, and the weighing bottle immediately after the sealing is measured.
(4) The sealed weighing bottle is left to stand at 40° C. and 30% RH for 24 hours.
(5) A total mass M2 of the measurement sample, the water, and the weighing bottle after being left to stand for 24 hours is measured.
(6) A moisture permeability Pl is calculated from the following formula (2):

$$Moisture Permeability P 1 = ( Total Mass M 1 - Total Mass M 2 ) / Predetermined Area S \qquad ( 2 )$$

The hardness (strength) of the cover member 11 can be appropriately designed to have a desirable magnitude, and, for example, may be from 10 through 40. When the hardness of the cover member 11 is within the above preferable range, the upper sheet 12, the electrode 20, and the second layer member 40 can readily deform in accordance with the movement of the skin 2 without being influenced by the cover member 11 when the skin 2 is extended by the body movements. The hardness (how hard it is) refers to Shore A hardness. In the present specification, the Shore A hardness refers to the hardness measured based on IS07619 (JIS K 6253). The Shore A hardness can be measured by a typical Shore A hardness meter.

### Upper Sheet

As illustrated in FIG. 3, the upper sheet 12 is adhered to the lower surface of the cover member 11. The upper sheet 12 has a through-hole 12a at a position facing the projection 111 of the cover member 11. Owing to the through-hole 12a, the sensor body 32 of the sensor portion 30 is housed in the housing space S, formed by the recess 111a at the inner surface of the cover member 11 and the through-hole 12a, without being blocked by the upper sheet 12.

The upper sheet 12 includes a jutting-out portion 12A projecting outward of the cover member 11 in a plan view, and is formed so as to have a shape larger than the cover member 11 outward of the cover member 11. That is, the jutting-out portion 12A is an outer peripheral portion of a first base 121 not covered by the cover member 11, and projects beyond the outer peripheral portion of the cover member 11 in a state in which the cover member 11 is attached.

The amount of jutting-out (length of projection) of the jutting-out portion 12A from the outer peripheral portion of the upper sheet 12 can be appropriately set to a desired amount, and, for example, may be about several millimeters, preferably from 3 mm through 10 mm, and more preferably from 5 mm through 7 mm. The amount of jutting-out of the jutting-out portion 12A may be a length of projection from the outer peripheral portion of the cover member 11. When the outer periphery of the cover member 11 is partially recessed, projected, or the like, in a plan view of the biological sensor 1, the amount of jutting-out of the jutting-out portion 12A may be the shortest distance.

The upper sheet 12 includes: the first base 121; a first adhesive layer 122 that is provided at one surface of the first base 121 facing the electrode 20 and to which the electrode 20 is attached; and an upper adhesive layer 123 that is provided at the surface (upper surface) of the first base 121 opposite to the surface directed toward the second layer member 40, which is the surface facing the electrode 20.

The jutting-out portion 12A may be formed by two layers, i.e., a jutting-out portion 121A of the first base 121 forming the upper sheet 12, and a jutting-out portion 122A of the first adhesive layer 122. It is enough that the jutting-out portion 12A may be formed by including the jutting-out portion 121A of the first base 121. For example, the jutting-out portion 12A may be formed only by the jutting-out portion 121A. The jutting-out portion 12A may be formed by three layers, i.e., the jutting-out portion 121A, the jutting-out portion 122A, and a jutting-out portion of the upper adhesive layer 123. In order to prevent adhesion to clothes or the like and adhesion of dust or the like, preferably, the jutting-out portion 12A does not include the jutting-out portion at the upper adhesive layer 123.

### First Base

As illustrated in FIG. 3, the first base 121 is provided on the attachment side that is the opening side of the cover member 11. As illustrated in FIG. 1, the first base 121 is formed in a sheet shape. The first base 121 may have flexibility, waterproofness, and moisture permeability. Because the first base 121 has flexibility, waterproofness, and moisture permeability, the first base 121 can be readily stretched in the state of contacting the skin 2. Thus, the state of contacting the skin 2 can be maintained, and also the entry of liquid into the gap between the first base 121 and the upper adhesive layer 123 can be suppressed. Further, water vapor derived from sweat or the like generated from the skin 2 can be released to the exterior of the biological sensor 1 through the first base 121. Therefore, the upper sheet 12 readily maintains adhesion durability.

As long as the first base 121 has flexibility, waterproofness, and moisture permeability, the first base 121 may be a non-porous body having no porous structure or may be a porous body having a porous structure. When the first base 121 is a non-porous body, the first base 121 is readily made thinner and the strength of the first base 121 is readily maintained, which is preferable. When the first base 121 is a porous body, water vapor derived from sweat or the like generated from the skin 2, to which the biological sensor 1 is attached, is readily released to the exterior of the biological sensor 1 through the first base 121, which is preferable.

As the non-porous body, a molded body formed into a sheet can be used.

The porous body may have a structure containing cells, such as open cells, closed cells, semi-closed cells, or the like. That is, the porous body may be a porous body produced through foam molding that forms communicating cells (a porous body having a communicating cell structure), may be a porous body produced through foam molding that forms closed cells (a porous body having a closed cell structure), or may be a porous body produced through foam molding that forms semi-closed cells (a porous body having a semi-closed cell structure). Of these, a porous body having a closed cell structure is preferable from the viewpoint of achieving a thinner film and maintaining strength while exhibiting higher waterproofness. As the porous body, a foamed sheet, a non-woven fabric sheet, or the like can be used.

As the material forming the first base 121, it is possible to use, for example, flexible materials including: thermoplastic resins, such as polyurethane-based resins, polystyrene-based resins, polyolefin-based resins, silicone-based resins, acrylic resins, vinyl chloride-based resins, polyester-based resins, and the like; thermoplastic elastomers; and the like.

Examples of the thermoplastic elastomer include polyurethane-based thermoplastic elastomers, polystyrene-based thermoplastic elastomers, polyolefin-based thermoplastic elastomers, polyester-based thermoplastic elastomers, polyvinyl chloride-based thermoplastic elastomers, polyamide-based thermoplastic elastomers, nitrile-based thermoplastic elastomers, nylon-based thermoplastic elastomers, fluororubber-based thermoplastic elastomers, polybutadiene-based thermoplastic elastomers, ethylene vinyl acetate-based thermoplastic elastomers, chlorinated polyethylene-based thermoplastic elastomers, styrene-butadiene block copolymers or hydrogenated products of the styrene-butadiene block copolymers, styrene-isoprene block copolymers or hydrogenated products of the styrene-isoprene block copolymers, and the like. These may be used alone or in combination. Of these, polyurethane-based thermoplastic elastomers are preferable.

When the first base 121 is a non-porous body, specifically, a polyurethane sheet, such as, for example, ESMER URS available from Nihon Matai Co., Ltd., can be used.

When the first base 121 is a porous body, specifically, a foamed sheet, such as, for example, FOLEC available from INOAC CORPORATION or a non-woven sheet, such as, for example, a medical patch base EW available from Japan Vilene Company, Ltd. may be used.

The first base 121 includes the jutting-out portion 121A. The jutting-out portion 121A may project at the outer peripheral portion of the first base 121 in the longitudinal direction and the transverse direction beyond the outer peripheral portions of the cover member 11 and the second layer member 40 in the longitudinal direction and the transverse direction. The jutting-out portion 121A may project beyond the outer peripheral portion of the first base 121 in the longitudinal direction or in the transverse direction.

The first base 121 has moisture permeability, and thus water vapor derived from sweat or the like can be efficiently released from the jutting-out portion 121A.

This can suppress accumulation of moisture, such as sweat and the like, between the first base 121 and the skin 2.

Therefore, it is possible to suppress skin rashes, and suppress peeling of the jutting-out portion 121A.

The first base 121 may be set to have higher stretchability than that of the cover member 11.

The moisture permeability of the first base 121 may be higher than that of the cover member 11. However, the moisture permeability of the first base 121 is 3,600 g/(m²·day) or less, preferably 3, 500 g/(m²·day) or less, and more preferably 2,000 g/(m²·day) or less. The lower limit of the moisture permeability of the first base 121 may be 100 g/(m²·day) or more. When the moisture permeability of the first base 121 is 3,600 g/(m²·day) or less, the entry of water vapor from the exterior is suppressed.

No particular limitation is imposed on the calculation method of the moisture permeability of the first base 121, and a typical method can be used. A measurement method similar to that for the moisture permeability of the cover member 11 may be used.

The thickness of the first base 121 can be appropriately set in accordance with the type of the first base 121 and the like, but is preferably larger than the thickness of the outer peripheral portion of the cover member 11. When the thickness of the first base 121 is larger than the thickness of the outer peripheral portion of the cover member 11, it is possible to reduce irritation caused by contact of the outer peripheral portion of the cover member 11 with the skin 2. For example, the thickness of the first base 121 is preferably from 10 um through 1.5 mm, and more preferably from 0.7 mm through 1.0 mm.

When the first base 121 is formed by a porous body, such as a foamed sheet, a non-woven fabric sheet, or the like, for example, the thickness of the first base 121 is preferably from 0.5 mm through 1.5 mm, and more preferably from 1.0 mm through 1.3 mm.

When the first base 121 is formed by a non-porous body, such as a polyurethane sheet or the like, for example, the thickness of the first base 121 is preferably from 10 μm through 300 μm, and more preferably from 30 μm through 200 μm.

As illustrated in FIG. 3, the first base 121 has a through-hole 121a at a position facing the projection 111 of the cover member 11. When the first adhesive layer 122 and the upper adhesive layer 123 are provided on the surface of the first base 121 other than the through-hole 121a, through-holes 122a and 123a can also be formed in the first adhesive layer 122 and the upper adhesive layer 123. The through-holes 121a, 122a, and 123a form the through-hole 12a.

### First Adhesive Layer

As illustrated in FIG. 3, the first adhesive layer 122 is attached to one surface of the first base 121 facing the electrode 20. The first adhesive layer 122 is positioned at a surface of the first base 121 facing the living body (−Z-axis direction), and has the function of adhering the skin 2 and the first base 121 to each other, the function of adhering the first base 121 and a second base 41 to each other, and the function of adhering the first base 121 and the electrode 20 to each other.

The first adhesive layer 122 may have moisture permeability. As such, as described below, water vapor derived from sweat or the like generated from the skin 2, to which the biological sensor 1 is attached, can be escaped to the first base 121 through the first adhesive layer 122, and can be released to the exterior of the biological sensor 1 through the first base 121. When the first base 121 has a cell structure as described above, water vapor can be released to the exterior of the biological sensor 1 through the first adhesive layer 122. This can prevent sweat or water vapor from accumulating at the interface between the skin 2, on which the biological sensor 1 is attached, and the first layer member 10. As a result, it is possible to prevent the adhesive strength of the first adhesive layer 122 from weakening due to the moisture accumulated at the interface between the skin 2 and the first adhesive layer 122, and prevent peeling of the biological sensor 1 off from the skin 2.

Preferably, the moisture permeability of the first adhesive layer 122 is, for example, 1 g/(m²·day) or more. The moisture permeability of the first adhesive layer 122 may be 10,000 g/(m²·day) or less. As long as the moisture permeability of the first adhesive layer 122 is 1 g/(m²·day) or more, when the first adhesive layer 122 is attached to the skin 2, sweat or the like delivered from the first adhesive layer 122 can be released toward the exterior. This can reduce the burden on the skin 2.

As the material forming the first adhesive layer 122, a material having pressure-sensitive adhesiveness may be used. As the material having pressure-sensitive adhesiveness, for example, an acrylic adhesive, a silicone-based adhesive, or the like can be used, and an acrylic adhesive is preferably used. As the acrylic adhesive, the acrylic polymers and the like described in Japanese Patent Application Laid-Open No. 2002-65841 are exemplified.

The first adhesive layer 122 may be double-sided adhesive tape formed of the above material.

A wave pattern may be formed on the surface of the first adhesive layer 122. This wavy pattern is formed by alternatingly forming an adhesive-applied portion, in which an adhesive is present, and an adhesive-free portion, in which the adhesive is absent. As the first adhesive layer 122, for example, double-sided adhesive tape having a wave pattern formed on a surface of the double-sided adhesive tape may be used. Since the first adhesive layer 122 has a wave pattern on the surface, it is possible to attach the adhesive to and around a protrusion of the surface and not to attach the adhesive to and around a recess of the surface. Thus, the surface of the first adhesive layer 122 includes both of portions, in which the adhesive is present, and portions, in which the adhesive is absent so that the adhesive can be sparsely located on the surface of the first adhesive layer 122. The moisture permeability of the first adhesive layer 122 tends to increase as the adhesive is thinner. Therefore, by forming the wave pattern on the surface of the first adhesive layer 122 and providing the surface of the first adhesive layer 122 with portions in which the adhesive is thinner, it is possible to enhance the moisture permeability while maintaining the adhesive strength, compared to a case in which the w pattern is not formed.

The widths of the adhesive-applied portion and the adhesive-free portion may be appropriately designed.

For example, the width of the adhesive-applied portion is preferably from 500 μm through 1,000 μm, and the width of the adhesive-free portion is preferably from 1,500 μm through 5,000 μm. When the widths of the adhesive-applied portion and the adhesive-free portion are within the above preferable ranges, the first adhesive layer 122 can exhibit excellent moisture permeability while maintaining the adhesive strength.

The thickness of the first adhesive layer 122 may be desirable set, and, for example, may be from 10 μm through 300 μm. When the thickness of the first adhesive layer 122 is from 10 μm through 300 μm, the biological sensor 1 can be reduced in thickness.

The first adhesive layer 122 includes the jutting-out portion 122A. The jutting-out portion 122A projects beyond the outer peripheral portion of the cover member 11, and thus the attachment area to the skin 2 can be increased compared to a case in which the upper sheet 12 is formed to have the same size as that of the cover member 11. As such, the attachment performance of the biological sensor 1 to the skin 2 can be enhanced.

Also, when the skin 2 is deformed by the body movements or the like of the subject with the biological sensor 1 being attached to the skin 2, the biological sensor 1 deforms so as to follow the deformation of the skin 2. At this time, the jutting-out portion 121A, which is positioned beyond the outer peripheral portion of the cover member 11, can suppress direct contact of the end of the outer peripheral portion of the cover member 11 with the skin upon the deformation of the skin 2. Therefore, it is possible to suppress the irritation of the skin 2 caused by the outer peripheral portion of the cover member 11, and hence suppress the occurrence of pain or itching of the skin 2.

When the skin 2 is extended by the body movements, the outer peripheral portion of the upper sheet 12 is extended while following the extension of the skin 2. At this time, the deformation of the first adhesive layer 122 of the upper sheet 12 and the deformation of the upper sheet 12 relax the stress applied to the cover member 11, thereby suppressing the deformation on the upper adhesive layer 123 side. That is, the jutting-out portion 12A of the upper sheet 12 can function as a buffer that absorbs the extension of the skin 2, and the extension of the skin 2 can be partially absorbed by the jutting-out portion 12A.

In this manner, it is possible to reduce the extension of the outer peripheral portion of the cover member 11 caused by the extension of the skin 2. This can suppress pulling of the skin 2 in a direction of shrinkage caused as the reaction force of the extension of the outer peripheral portion of the cover member 11 in accordance with the extension of the skin 2, thereby enabling suppressing the occurrence of pain or itching due to the body movements of the skin 2 to which the outer peripheral portion of the biological sensor 1 is attached. As a result, the sensation felt by the subject during the attachment of the biological sensor 1 to the skin 2 can be improved.

Also, the contact area of the first adhesive layer 122 of the upper sheet 12 with the skin 2 can be increased compared to a case in which the upper sheet 12 is formed to have the same size as that of the cover member 11. Thus, the adhesive strength of the first adhesive layer 122 can be weaker than in the case in which the upper sheet 12 is formed to have the same size as that of the cover member 11. This can reduce the adhesive strength per unit area of the first adhesive layer 122. Thus, the biological sensor 1 can be readily peeled off from the skin 2 without degrading the adhesive performance of the biological sensor 1 to the skin 2. For example, the biological sensor 1 can be peeled off from the skin 2 without using any tool, such as a remover or the like and without causing the subject to feel pain.

In the present embodiment, the jutting-out portion 12A is entirely projected beyond the outer peripheral portion of the cover member 11. However, only a part of the jutting-out portion 12A may be projected beyond the outer peripheral portion of the cover member 11. That is, the jutting-out portion 12A may be provided only at a position at which the biological sensor 1 is likely to be peeled off from the skin 2 due to the body movements or the like.

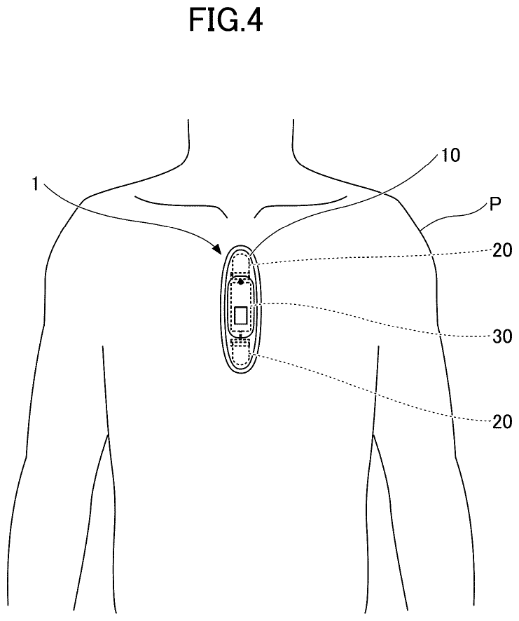

For example, when the biological sensor 1 is attached to a living body P such that a flat portion 112A of the cover member 11 is positioned closer to the belly of the living body P (see FIG. 4), displacement of the flat portion 112A due to the body movements is likely to be greater than displacement of a flat portion 112B, and the flat portion 112A of the biological sensor 1 is more likely peeled off than is the flat portion 112B of the biological sensor 1. Therefore, the jutting-out portion 12A is preferably provided at least at the end of the cover member 11 on the flat portion 112A side.

Further, displacement of the ends of the biological sensor 1 in the longitudinal direction (Y-axis direction) due to the body movements is likely to be greater than displacement of the ends of the biological sensor 1 in the width direction (X-axis direction). Thus, the ends of the biological sensor 1 in the longitudinal direction (Y-axis direction) are more likely to be peeled off. Therefore, the jutting-out portion 12A is preferably provided only at both ends in the longitudinal direction (Y-axis direction) of the biological sensor 1.

By providing the jutting-out portion 12A at portions of the biological sensor 1 that are more likely to be peeled off, as described above, the force pressed against the skin by the cover member 11 in response to deformation of the skin 2 due to the body movements can be dispersed in the upper sheet 12. Thus, it is possible to suppress peeling of the biological sensor 1 caused by the reaction force from the skin against the force of the cover member 11 pressed against the jutting-out portion 12A.

### Upper Adhesive Layer

As illustrated in FIG. 3, the upper adhesive layer 123 is attached to the surface (upper surface) of the first base 121 opposite to the surface facing the electrode 20. The upper adhesive layer 123 is attached to the upper surface of the first base 121 and at a position corresponding to the flat surface on the attachment side (−Z-axis direction) of the cover member 11. The upper adhesive layer 123 has the function of adhering the first base 121 and the cover member 11 to each other.

As the material forming the upper adhesive layer 123, a biocompatible material is used. For example, as the biocompatible material, an acrylic adhesive, a silicone-based adhesive, silicone tape, or the like, can be used. It is preferable to use a silicone-based adhesive.

The thickness of the upper adhesive layer 123 may be appropriately set, and, for example, may be from 10 μm through 300 μm.

### Moisture Permeable Film

As illustrated in FIGS. 2 and 3, the moisture permeable film 13 may be provided in the recess 111a of the cover member 11 so as to cover the through-hole 111b. The moisture permeable film 13 may be fixed to the recess 111a of the cover member 11 with an adhesive 14. The adhesive 14 may be provided along the outer peripheral ends of the upper surface of the moisture permeable film 13. Note that the adhesive 14 may be provided only at corners of the outer periphery of the upper surface of the moisture permeable film 13 or only at several positions on the outer periphery of the upper surface of the moisture permeable film 13.

The moisture permeability of the moisture permeable film 13 may be adjusted such that the above second permeating amount M2 is smaller than the above first moisture permeating amount M1.

The thickness of the moisture permeable film 13 is not particularly limited and may be any appropriate thickness.

As long as the adhesive 14 can fix the moisture permeable film 13 to the cover member 11, the adhesive 14 may be, for example, a commonly used adhesive.

Moreover, the moisture permeable film 13 may be provided by being fixed to an inner wall of the through-hole 111b with the adhesive 14 or the like so as to cover the through-hole 111b.

The moisture permeable film 13 is required to be a film permeable to water vapor, and, for example, a moisture permeable sheet or the like is used as the moisture permeable film 13.

### Electrode

As illustrated in FIG. 3, the electrode 20 may be attached to the lower surface of the first adhesive layer 122 on the attachment side (−Z-axis direction) in a state in which a part of the electrode 20 on the sensor body 32 side is connected to interconnects 331A and 331B and is held between the first adhesive layer 122 and a lower adhesive layer 42. The electrode 20 contacts the living body at a portion that is not held between the first adhesive layer 122 and the lower adhesive layer 42. When the biological sensor 1 is attached to the skin 2, the electrode 20 contacts the skin 2, thereby enabling detecting biological signals. The electrode 20 may be embedded in a second base 41 in a state in which the electrode 20 is exposed so as to be able to contact the skin 2.

The electrode 20 is formed by a pair of electrodes 20A and 20B. As illustrated in FIG. 3, the electrode 20A is disposed on the left-hand side in the drawing, and the electrode 20B is disposed on the right-hand side in the drawing. One end (inner side) of the electrode 20A in the longitudinal direction (Y-axis direction) contacts a terminal 332A, and one end (inner side) of the electrode 20B in the longitudinal direction (Y-axis direction) contacts a terminal 332B. The pair of electrodes 20A and 20B have substantially the same shape.

The one end of the electrode 20A that contacts the terminal 332A of the sensor portion 30 is referred to as a facing portion 201A, and the one end of the electrode 20B that contacts the terminal 332B of the sensor portion 30 is referred to as a facing portion 201B. A portion of the electrode 20A that does not contact the terminal 332A (the other end (outer side) in the longitudinal direction (Y-axis direction)) is referred to as an exposed portion 202A, and a portion of the electrode 20B that does not contact the terminal 332B (the other end (outer side) in the longitudinal direction (Y-axis direction)) is referred to as an exposed portion 202B.

The electrode 20 may have any shape, such as a sheet shape.

No particular limitation is imposed on the shape of the electrode 20 in a plan view. The electrode 20 may be designed to have a shape that is appropriate in accordance with applications or the like. As illustrated in FIG. 2, the electrode 20 may be formed such that in a plan view, the one end, i.e., the facing portion 201A or 201B, is formed in a rectangular shape, and the other end, i.e., the exposed portion 202A or 202B, is formed in an arc shape.

The electrode 20 can be formed using a cured product of a conductive composition containing a conductive polymer and a binder resin, a metal, an alloy, or the like. Of these, it is preferable to form the electrode 20 using a cured product of a conductive composition from the viewpoint of safety of the living body, such as, for example, avoiding an allergic reaction or the like occurring when the electrode 20 is applied to the living body. The electrode 20 for use may be an adhesive electrode sheet that is obtained by forming a cured product of a conductive composition in the form of a sheet.

As the conductive polymer, for example, it is possible to use a polythiophene-based conductive polymer, a polyaniline-based conductive polymer, a polyacetylene-based conductive polymer, a polypyrrole-based conductive polymer, a polyphenylene-based conductive polymer, a derivative of the above-listed polymers, a composite of the above-listed polymers, or the like. Of these, it is preferable to use composites in which polythiophene is doped with polyaniline as a dopant. Of the composites of polythiophene and polyaniline, it is more preferable to use PEDOT/PSS in which poly (3,4-ethylenedioxythiophene) (also referred to as PEDOT), which is polythiophene, is doped with polystyrene sulfonic acid (poly 4-styrenesulfonate; PSS), which is polyaniline. This is because of low contact impedance with the living body and high conductivity.

The binder resin for use may be a water-soluble polymer, a water-insoluble polymer, or the like. The water-soluble polymer for use may be a hydroxyl group-containing polymer, such as polyvinyl alcohol (PVA), modified PVA, and the like.

The conductive composition may appropriately contain various typical additives, such as a crosslinking agent, a plasticizer, and the like, in a desired ratio. Examples of the crosslinking agent include aldehyde compounds, such as sodium glyoxylate and the like. Examples of the plasticizer include glycerin, ethylene glycol, propylene glycol, and the like.

The metal and the alloy for use may be typical metals and alloys, such as Au, Pt, Ag, Cu, Al, and the like.

The thickness of the electrode 20 may be an appropriate height and may be, for example, from 10 μm through 100 μm. When the thickness of the electrode 20 is within the above preferable range, the electrode 20 can have sufficient strength and flexibility.

The thickness of the electrode 20 is a length of the electrode 20 in a direction perpendicular to the surface of the electrode 20. The thickness of the electrode 20 is, for example, a thickness as measured at a given site in a cross section of the electrode 20. When measurement is performed at a plurality of given sites, the average value of the thicknesses measured at the plurality of given sites may be used as the thickness of the electrode 20.

### Sensor Portion

As illustrated in FIG. 2, the sensor portion 30 has a flexible substrate 31, the sensor body 32, and connection portions 33A and 33B connected to the sensor body 32.

The flexible substrate 31 is a resin substrate on which various parts configured to obtain biological information are mounted. The sensor body 32 and the connection portions 33A and 33B are disposed on the flexible substrate 31.

As illustrated in FIG. 2, the sensor body 32 includes a part-mounting portion 321, serving as a controller, and a battery-mounting portion 322, and obtains biological information.

The part-mounting portion 321 includes various parts mounted on the flexible substrate 31, and obtains biological information. These parts are: a CPU and an integrated circuit configured to process biological signals obtained from the living body and generate biological signal data; a switch configured to start-up the biological sensor 1; a flash memory configured to store biological signals; a light-emitting element; and the like. An example of a circuit formed of these parts is omitted. The part-mounting portion 321 is driven by power supplied from a battery 34 mounted on the battery-mounting portion 322.

The part-mounting portion 321 is configured to perform wired or wireless transmission to external devices, such as a drive identifier configured to confirm initial driving, a reader configured to read biological information from the biological sensor 1, and the like.

The battery-mounting portion 322 is disposed between the connection portion 33A and the part-mounting portion 321, and is configured to supply power to an integrated circuit or the like mounted on the part-mounting portion 321. As illustrated in FIG. 2, the battery 34 is mounted on the battery-mounting portion 322.

In the longitudinal direction (Y-axis direction) of the sensor body 32, the connection portions 33A and 33B include: the interconnects 331A and 331B connected to the sensor body 32; and the terminals 332A and 332B provided at the distal ends of the interconnects 331A and 331B and connected to the electrode 20.

As illustrated in FIG. 3, one end of the interconnect 331A or 331B is connected to the electrode 20. As illustrated in FIG. 3, the other end of the interconnect 331A is connected to the switch or the like mounted on the part-mounting portion 321 along the outer periphery of the sensor body 32. The other end of the interconnect 331B is connected to the switch and the like mounted on the part-mounting portion 321. Note that the interconnects 331A and 331B may be formed on an interconnect layer on a front or a back surface of the flexible substrate 31.

The terminal 332A or 332B is disposed in a state in which one end of the terminal 332A or 332B is connected to the interconnect 331A or 331B, and the upper surface of the other end of the terminal 332A or 332B is in contact with the electrode 20 and is held between the first layer member 10 and the second layer member 40.

A publicly known battery can be used as a battery 34. For example, a coin-type battery, such as CR 2025 or the like, can be used as the battery 34.

### Second Layer Member

As illustrated in FIG. 3, the second layer member 40 is attached to a surface (lower surface) of the first layer member 10 opposite to the surface that projects, and has the attachment surface to be attached to the skin 2. The second layer member 40 is provided on an attachment surface side of the electrode 20 and the sensor portion 30. The second layer member 40 is a support substrate on which the sensor portion 30 is provided, and also forms a part of the attachment surface to the skin 2.

As illustrated in FIGS. 1 and 2, the outer shape of the second layer member 40 at both sides in the width direction (X-axis direction) may be formed into substantially the same shape as the outer shape of the first layer member 10 at both sides in the width direction (X-axis direction). The length (Y-axis direction) of the second layer member 40 may be formed to be shorter than the length (Y-axis direction) of the cover member 11 and the upper sheet 12. The second layer member 40 may be provided such that the electrode 20 is exposed at the lower surface of the first layer member 10. As illustrated in FIG. 3, both of the longitudinal ends of the second layer member 40 are located at positions that hold the interconnects 331A and 331B of the sensor portion 30 between the second layer member 40 and the upper sheet 12, and that overlap with a part of the electrode 20.

The second layer member 40 includes the second base 41, the lower adhesive layer 42 provided on the upper surface of the second base 41, and a second adhesive layer 43 provided on the lower surface of the second base 41. The second base 41, the lower adhesive layer 42, and the second adhesive layer 43 may be formed in the same shape in a plan view. The attachment surface to the skin 2 is formed by the second adhesive layer 43 of the second layer member 40 and the electrode 20. In accordance with the area of the electrode 20 and the second adhesive layer 43, the waterproofness and the moisture permeability are different from position to position on the attachment surface, and thus the adhesiveness can be made different. Therefore, it is possible to enable the waterproofness, the moisture permeability, and the adhesiveness to differ in accordance with the area of the attachment surface of the second adhesive layer 43.

### Second Base

The second base 41 can be formed of a flexible resin having appropriate stretchability, flexibility, and toughness. As a material forming the second base 41, for example, it is possible to use a thermoplastic resin including: a polyester-based resin, such as polyethylene terephthalate (PET), polybutylene terephthalate, polytrimethylene terephthalate, polyethylene naphthalate, polybutylene naphthalate, or the like; an acrylic resin, such as polyacrylic acid, polymethacrylic acid, polymethyl acrylate, polymethyl methacrylate (PMMA), polyethyl methacrylate, polybutyl acrylate, or the like; a polyolefin-based resin, such as polyethylene, polypropylene, or the like; a polystyrene-based resin, such as polystyrene, an imide-modified polystyrene, an acrylonitrile-butadiene-styrene (ABS) resin, an imide-modified ABS resin, a styrene-acrylonitrile copolymer (SAN) resin, an acrylonitrile ethylene-propylene-diene styrene (AES) resin, or the like; a polyimide-based resin; a polyurethane-based resin; a silicone-based resin; a polyvinyl chloride-based resin, such as polyvinyl chloride, a vinyl chloride-vinyl acetate copolymer resin, or the like. Of these, a polyolefin-based resin and PET are preferably used. These thermoplastic resins have waterproofness that does not permit permeation of water and water vapor (low in water permeability). Therefore, when the second base 41 is formed of any of these thermoplastic resins, it is possible to suppress the entry of sweat or water vapor generated from the skin 2 into the flexible substrate 31 of the sensor portion 30 through the second base 41 in a state in which the biological sensor 1 is attached to the skin 2 of the living body.

The second base 41 is preferably formed in a flat-plate shape because the sensor portion 30 is disposed on the upper surface via the lower adhesive layer 42.

The thickness of the second base 41 can be appropriately selected and, for example, may be from 1 μm through 300 μm.

### Lower Adhesive Layer

As illustrated in FIG. 3, the lower adhesive layer 42 is provided on the upper surface of the second base 41 on the cover member 11 side (+Z-axis direction), and the sensor portion 30 is adhered to the lower adhesive layer 42. Both longitudinal ends of the lower adhesive layer 42 of the second layer member 40 are provided at positions that face the facing portions 201A and 201B of the electrode 20. As such, the facing portions 201A and 201B of the electrode 20 and the terminals 332A and 332B can be held between the upper sheet 12 and the second layer member 40 in a state of being compressed, and the electrode 20 and the terminals 332A and 332B can be electrically connected. The lower adhesive layer 42 can be formed of a material similar to that of the second adhesive layer 43 described below, and details will be omitted. The lower adhesive layer 42 does not necessarily need to be provided, and may be absent.

### Second Adhesive Layer

As illustrated in FIG. 3, the second adhesive layer 43 is provided on the lower surface of the second base 41 on the attachment side (−Z-axis direction) and contacts the skin 2.

The second adhesive layer 43 preferably has pressure-sensitive adhesiveness. By virtue of the pressure-sensitive adhesiveness of the second adhesive layer 43, the biological sensor 1 can be readily attached to the skin 2 by pressing the biological sensor 1 against the skin 2 of the living body.

No particular limitation is imposed on the material of the second adhesive layer 43 as long as the material has pressure-sensitive adhesiveness, and the material is a biocompatible material or the like. Examples of the material forming the second adhesive layer 43 include acrylic pressure-sensitive adhesives, silicone-based pressure-sensitive adhesives, and the like. Silicone-based pressure-sensitive adhesives are preferable.

The acrylic pressure-sensitive adhesive preferably contains an acrylic polymer as a main component. The acrylic polymer can function as a pressure-sensitive adhesive component. The acrylic polymer for use is a polymer obtained through polymerization of a monomer component containing a (meth)acrylic acid ester, such as isononyl acrylate, methoxyethyl acrylate, or the like, as a main component and a monomer copolymerizable with a (meth)acrylic acid ester, such as acrylic acid or the like, as an optional component.

The acrylic pressure-sensitive adhesive preferably further contains a carboxylic acid ester. The carboxylic acid ester functions as an adjuster for pressure-sensitive adhesive strength that adjusts the pressure-sensitive adhesive strength of the second adhesive layer 43 by reducing the pressure-sensitive adhesive strength of the acrylic polymer. As the carboxylic acid ester, a carboxylic acid ester compatible with the acrylic polymer can be used. As the carboxylic acid ester, fatty acid triglyceride or the like can be used.

If necessary, the acrylic pressure-sensitive adhesive may contain a crosslinking agent. The crosslinking agent is a crosslinking component that crosslinks the acrylic polymer. Examples of the crosslinking agent include polyisocyanate compounds (polyfunctional isocyanate compounds), epoxy compounds, melamine compounds, peroxide compounds, urea compounds, metal alkoxide compounds, metal chelate compounds, metal salt compounds, carbodiimide compounds, oxazoline compounds, aziridine compounds, amine compounds, and the like. Of these, polyisocyanate compounds are preferable. These crosslinking agents may be used alone or in combination.

The second adhesive layer 23 may be double-sided tape formed of the above material.

The second adhesive layer 43 preferably has excellent biocompatibility. For example, when the second adhesive layer 43 is subjected to a keratin peeling test, a keratin-peeled area percentage is preferably from 0% through 50%. When the keratin-peeled area percentage is in the range of from 0% through 50%, the burden on the skin 2 can be suppressed even if the second adhesive layer 43 is attached to the skin 2.

The second adhesive layer 43 preferably has moisture permeability. Water vapor and the like generated from the skin 2, to which the biological sensor 1 is attached, can be escaped toward the upper sheet 12 through the second adhesive layer 43. Also, as described above, the upper sheet 12 has a structure having cells. Thus, water vapor can be released to the exterior of the biological sensor 1 through the second adhesive layer 43. This can prevent sweat or water vapor from accumulating at the interface between the skin 2, to which the biological sensor 1 is attached, and the second adhesive layer 43. As a result, it is possible to prevent the adhesive strength of the second adhesive layer 43 from weakening due to the moisture accumulated at the interface between the skin 2 and the second adhesive layer 43, and prevent peeling of the biological sensor 1 off from the skin 2.

Preferably, the moisture permeability of the second adhesive layer 43 is, for example, from 300 g/(m²·day) through 10,000 g/(m²·day). When the moisture permeability of the second adhesive layer 43 is in the above preferable range, even if the second adhesive layer 43 is attached to the skin 2, sweat or the like generated from the skin 2 can be appropriately released toward the exterior through the second adhesive layer 43. This can reduce the burden on the skin 2.

No limitations are imposed on the method of measuring the moisture permeability of the second adhesive layer 43, and a typical measurement method may be used.

The moisture permeability of the second adhesive layer may be measured, for example, in accordance with the following procedure.

### 1. Preparation of Samples

The following samples are prepared.
(1) PET workpiece: An opening of 20 mm in diameter is formed at the central portion of PET of 50 mm in length and 50 mm in width.
(2) Double-sided tape workpiece: Double-sided tape (No. 5000NS, available from Nitto Denko Corporation) is attached to a separator and processed into a ring shape of 30 mm in diameter and a hole of 20 mm in diameter at the central portion.
(3) Sample workpiece: Damplon tape is attached to an upper surface of a sample sheet with a roller and processed into a disk shape of 30 mm in diameter.

### 2. Measurement of Moisture Permeability

(1) 10 ml of water (ion exchanged water) is charged into a moisture permeability cup (diameter of opening: 38 mm, height: 40 mm).
(2) The opening of the PET workpiece is laid over the hole of the double-sided tape workpiece, and the double-sided tape workpiece is attached to the PET workpiece such that there is no gap between the PET workpiece and the double-sided tape workpiece. Then, a workpiece 1 is produced.
(3) The sample workpiece is attached to the double-sided tape workpiece of the workpiece 1 such that there is no gap between the double-sided tape workpiece and the sample workpiece. Then, a workpiece 2 is produced.
(4) The workpiece 2 is placed on the moisture permeability cup such that the center of the workpiece 2 coincides with the center of the moisture permeability cup. Then, vinyl tape (adhesive tape) is wrapped around the end of the workpiece 2 and the side surface of the moisture permeability cup to fix the end of the workpiece 2 to the side surface of the moisture permeability cup. The moisture permeability cup is sealed, and then a workpiece 3 is produced.
(5) A mass M1 (total mass of the PET workpiece, the double-sided tape workpiece, the sample workpiece, the vinyl tape, the moisture permeability cup, and the water) of the workpiece 3 is measured.
(6) The workpiece 3 is put into a thermostatic bath and left to stand for 24 hours under conditions of 40° C. and 30% RH.
(7) After being left to stand for 24 hours, the workpiece 3 is taken out from the thermostatic bath, and a mass M2 of the workpiece 3 is measured after being left to stand for 24 hours.
(8) An evaporation amount after being left to stand for 24 hours is calculated from the following formula (3) as a moisture permeability P2:

$$Moisture Permeability P 2 [ g ] = ( Mass M 2 - Mass M 1 ) \qquad ( 3 )$$

The thickness of the second adhesive layer 43 can be appropriately selected, and is preferably from 10 μm through 300 μm. When the thickness of the second adhesive layer 43 is from 10 μm through 300 μm, the biological sensor 1 can become thinner.

As illustrated in FIGS. 1 and 2, when the biological sensor 1 is not in use, a release liner 50 is preferably attached to the surfaces of the electrode 20 and the second base 41 to be attached to the skin 2 until use in order to protect the electrode 20 and the second layer member 40. Upon use, the release liner 50 is peeled off from the electrode 20 and the second layer member 40, and then the attachment surface of the biological sensor 1 can be attached to the skin 2. When the release liner 50 is attached to the attachment surface, the adhesive strength of the electrode 20 and the second layer member 40 can be maintained, for example, even if the biological sensor 1 is stored for a long time. Therefore, by peeling off the release liner 50 from the second layer member 40 and the electrode 20 upon use, the attachment surface can be reliably attached to the skin 2 for use.

No particular limitation is imposed on a production method of the biological sensor 1. However, the biological sensor 1 can be produced by any appropriate method. An example of the production method of the biological sensor 1 will be described.

The first layer member 10, the electrode 20, the sensor portion 30, and the second layer member 40 illustrated in FIGS. 1 and 2 are provided. No particular limitation is imposed on production methods of the first layer member 10, the electrode 20, the sensor portion 30, and the second layer member 40 as long as they can be produced. The first layer member 10, the electrode 20, the sensor portion 30, and the second layer member 40 can be produced by any appropriate methods.

After providing the first layer member 10, the electrode 20, the sensor portion 30, and the second layer member 40 that form the biological sensor 1 illustrated in FIG. 1, the sensor portion 30 is placed on the second layer member 40. Subsequently, the first layer member 10, the electrode 20, the sensor portion 30, and the second layer member 40 are stacked in the order from the first layer member 10 side toward the second layer member 40 side. In this manner, the biological sensor 1 illustrated in FIG. 1 is obtained.

FIG. 4 is an explanatory view illustrating the biological sensor 1 of FIG. 1 attached to the chest of a subject P. As illustrated in FIG. 4, for example, the biological sensor 1 is attached to the skin of the subject P in a state in which the longitudinal direction (Y-axis direction) is aligned with the sternum of the subject P, and one electrode 20 faces upward and the other electrode 20 faces downward. When the biological sensor. is attached to the skin of the subject P by the effect of the second adhesive layer 43 of FIG. 2, the biological sensor 1 obtains biological signals, such as an electrocardiogram signal and the like, from the subject P via the electrode 20 in a state in which the electrode 20 is compressed to the skin of the subject P. The biological sensor 1 stores the obtained biological signal data in a non-volatile memory, such as a flash memory or the like, mounted on the part-mounting portion 321.

In this manner, the biological sensor 1 includes the first layer member 10, the sensor portion 30, the second layer member 40, and the housing space S, and the first permeating amount M1 is made smaller than the second permeating amount M2. The first permeating amount M1 is the permeating amount of the water vapor entering the housing space S through the second layer member 40 at the first temperature, and the second permeating amount M2 is the permeating amount of the water vapor exiting from the housing space S through the first layer member 10 at the second temperature T2.

The second layer member 40 is a member to contact the skin 2, and the first layer member 10 is a member to contact the exterior. When the biological sensor 1 is in use, the second layer member 40 is warmed to, for example, about 36° C., which is a temperature close to the body temperature, and the first layer member 10 is cooled to, for example, about 30° C., which is a temperature lower than the body temperature. Therefore, it is easier for the water vapor to pass through around the skin 2 where the temperature is higher, and it is more difficult for the water vapor to pass through around the cover member 11 of the first layer member 10 where the temperature is lower.

When an amount of the water vapor entering the housing space S from the skin 2 through the second layer member 40 is equal to or greater than an amount of the water vapor exiting from the housing space S through the first layer member 10, the amount of the water vapor in the housing space S tends to increase, and the water vapor tends to adhere to the sensor body 32 constituting the sensor portion 30 inside the housing space S, causing dew condensation. For example, when the temperature is warm around the skin 2 and the temperature at the outer side decreases, the water vapor moves more actively from the second layer member 40, which is a member to directly contact the skin 2, into the housing space S, and the water vapor moves less actively to exit from the housing space S to the outer side through the first layer member 10. Therefore, the water vapor tends to be accumulated inside the housing space S and become water droplets, causing dew condensation on the sensor body 32.

According to the biological sensor 1, by making the first permeating amount M1 smaller than the second moisture permeating amount M2, the accumulation of the water vapor derived from sweat generated from the skin 2 is suppressed inside the housing space S from the second layer member 40, and the water vapor is easily released from the housing space S to the exterior through the first layer member 10. Thus, the biological sensor 1 can reduce dew condensation onto the sensor body 32.

Since the biological sensor 1 can suppress the occurrence of a failure in the sensor body 32 by reducing dew condensation onto the sensor body 32, it is possible to stably measure biological signals.

In addition, the biological sensor 1 can suppress the accumulation of the water vapor between the skin 2 and the attachment surface of the biological sensor 1 even when the biological sensor 1 is attached to the skin 2 of the subject for a long period of time. Thus, the biological sensor 1 can reduce the accumulation of the water vapor derived from sweat or the like between the skin 2 and the attachment surface of the biological sensor 1 so as not to cause discomfort, such as itching and pain, for the subject. Therefore, it is possible to maintain a good attachment sensation.

According to the biological sensor 1, the first layer member 10 includes the cover member 11 and the upper sheet 12, and the upper sheet 12 can include the first base 121. The first layer member 10 can be constituted by stacking the cover member 11 and the first base 121.

Therefore, even if the first layer member 10 is constituted by a plurality of different members, the biological sensor 1 can form the housing space S enclosing the sensor body 32 in the first layer member 10. This enables the water vapor derived from sweat generated from the skin 2 to be easily released from the housing space S to the exterior through the first layer member 10, reducing dew condensation on the sensor body 32.

The biological sensor 1 includes the through-hole 111b at the cover member 11 of the first layer member 10 and can include the moisture permeable film 13 so as to cover the through-hole 111b. This enables the biological sensor 1 to increase the permeating amount of the cover member 11 so that the water vapor that has passed through from the second layer member 40 into the housing space S can be more easily released from the cover member 11 to the exterior. Therefore, since the biological sensor 1 can more easily make the first permeating amount M1 smaller than the second permeating amount M2, dew condensation onto the sensor body 32 can be further reduced.

The biological sensor 1 can have the second adhesive layer 43 on the surface of the second layer member 40 opposite to the first layer member 10. This enables the second layer member 40 to be attached to the skin 2 through the second adhesive layer 43 so that the biological sensor 1 can further lower the contact impedance of the electrode 20 with the surface of the skin 2 to further suppress the generation of noise as well as to be further stably attached to the skin 2. Therefore, the biological sensor 1 can further enhance detection accuracy of the biological signals and further stably maintain the attachment performance to the skin 2 during use.

The biological sensor 1 can have the electrode 20 on the surface of the first layer member 10 directed toward the second layer member 40 so as to be connected to the sensor body 32. This enables the electrode 20 to contact the surface of the skin 2 so that the biological sensor 1 can detect biological signals through the electrode 20 during use.

The biological sensor 1 can have the electrode 20 on the surface of the first layer member 10 directed toward the second layer member 40 as well as the first adhesive layer 122 on the surface of the first base 121 directed toward the second layer member 40. Since the first adhesive layer 122 has adhesiveness, the electrode 20 can contact the surface of the skin 2 in a state of being stably attached to the first layer member 10 by the first adhesive layer 122. Therefore, when the biological sensor 1 detects a biological signal through the electrode 20 by bringing the electrode 20 into contact with the surface of the skin 2, the biological sensor 1 can lower the contact impedance of the electrode 20 with the surface of the skin 2 to suppress the generation of noise as well as be further stably attached to the skin 2. Therefore, the biological sensor 1 can enhance detection accuracy of a biological signal with the electrode 20 as well as stably maintain the attachment performance to the skin 2 during use.

The biological sensor 1 can provide the second layer member 40 so as to expose the electrode 20 on the surface opposite to the cover member 11 of the first base 121. Thus, when the biological sensor 1 detects a biological signal through the electrode 20, the electrode 20 can be brought into contact with the surface of the skin 2. Therefore, the biological sensor 1 can reliably detect the biological signal during use.

The biological sensor 1 can provide the jutting-out portion 12A on the upper sheet 12. The jutting-out portion 12A can have the jutting-out portion 121A on the first base 121 and the jutting-out portion 122A on the first adhesive layer 122. This enlarges the attachment area of the first adhesive layer 122 to the skin 2 so that the biological sensor 1 can be hardly peeled off from the skin 2. Thus, the biological sensor 1 can be stably attached to the skin 2 for a long period of time compared to a case in which the first base 121 and the upper sheet 12 are formed to have the same size as the cover member 11. Therefore, the biological sensor 1 can increase the measurement time for biological information.

By providing the jutting-out portion 12A in the upper sheet 12, the biological sensor 1 can suppress direct contact of the outer peripheral edge of the cover member 11 with the skin 2 in accordance with the deformation of the skin 2 due to the body movements. This can reduce irritation (occurrence of pain) to the skin 2 caused by the outer periphery of the cover member 11. In particular, when the first base 121 has high flexibility, even if the outer peripheral edge of the cover member 11 is directed to the skin 2 due to the deformation of the skin 2, the biological sensor 1 can disperse, in the upper sheet 12, the compressing force of the outer peripheral edge of the cover member 11 against the skin 2. Therefore, it is possible to reduce the irritation to the skin 2.

The jutting-out portion 121A can function as a buffer that absorbs the extension of the skin 2. This can suppress pulling of the skin 2 in a direction of shrinkage caused as the reaction force of the extension of the outer peripheral portion of the cover member 11 in accordance with the extension of the skin 2. Therefore, the biological sensor 1 can suppress the occurrence of body movement-derived pain in the skin 2 to which the outer peripheral portion of the biological sensor 1 is attached. As a result, the sensation felt during the attachment of the biological sensor 1 can be improved.

The jutting-out portion 121A can disperse, in the upper sheet 12, the compressing force of the cover member 11 against the skin 2 in accordance with the deformation of the skin 2 due to the body movements.

Therefore, it is possible to suppress peeling of the biological sensor 1 caused by the reaction force from the skin 2 against the compressing force of the cover member 11. This allows the biological sensor 1 not to be appreciably peeled off from the skin 2, and thus can suppress reduction in the adhesive strength of the first adhesive layer 122 of the upper sheet 12. As a result, the biological sensor 1 can reduce the irritation to the skin 2 caused by the first adhesive layer 122.

The jutting-out portion 12A includes the jutting-out portion 122A of the first adhesive layer 122. Thus, the contact area of the skin 2 with the first adhesive layer 122 can be increased. This can attach the biological sensor 1 to the skin 2 even if the adhesive strength of the first adhesive layer 122 is weakened.

Therefore, the biological sensor 1 can be readily peeled off from the skin 2 after use without causing the living body P to feel pain.

The biological sensor 1 can have the upper adhesive layer 123 on the upper surface of the first base 121. Since the cover member 11 and the first base 121 can be joined through the upper adhesive layer 123, the first layer member 10 can form the housing space S enclosing the sensor body 32 while suppressing the separation of the cover member 11 and the first base 121. Therefore, even if the first layer member 10 is constituted by a plurality of different members, the biological sensor 1 can form the housing space S while suppressing the formation of a gap between the members constituting the first layer member 10, and enables the water vapor derived from sweat generated from the skin 2 to be easily released from the housing space S to the exterior through the first layer member 10, reducing dew condensation on the sensor body 32.

The first base 121 of the biological sensor 1 can be formed of a polyurethane-based thermoplastic elastomer. When the first base 121 is formed of a polyurethane-based thermoplastic elastomer, and the thickness of the first base 121 is adjusted to an appropriate thickness, it becomes easy to control the permeating amount and the adhesive strength of the first base 121 to the skin 2. Therefore, the biological sensor 1 can easily and stably maintain the attachment performance to the skin 2 even when the surface of the skin 2 is deformed due to the body movement.

The biological sensor 1 can form the attachment surface to the skin 2 by the first layer member 10, the electrode 20, and the second layer member 40. Thus, the thickness of the biological sensor 1 can be reduced.

Therefore, the biological sensor 1 can be reduced in size and can reduce the contact impedance with the surface of the skin 2 as well as be stably attached to the skin 2.

As described above, the biological sensor 1 can stably measure biological information from the skin 2 during use for a long period of time. Thus, the biological sensor 1 can be effectively used as an attachable biological sensor that is attached to the skin 2 of a human or the like for use. For example, the biological sensor 1 exhibits high detection sensitivity of an electrocardiogram when attached to the skin of the living body or the like. Thus, the biological sensor 1 can be suitably used, for example, in a wearable device for health care that requires a high effect of suppressing noise generated in the electrocardiogram.

Although the embodiments have been described above, the above embodiments are merely illustrative, and the present invention is not limited to the above embodiments. The above embodiments can be practiced in various other forms, and various combinations, omissions, substitutions, changes, and the like can be made without departing from the gist of the invention. These embodiments and modifications are encompassed in the scope and gist of the invention, and included in the scope equivalent to the inventions recited in the claims.

Embodiments of the present invention are, for example, as follows.

<1> A biological sensor to be attached to a living body, the biological sensor including:
a sensor body configured to obtain biological information;
a first layer member having a projecting shape so as to cover the sensor body;
a second layer member that is attached to a surface of the first layer member and includes an attachment surface to be attached to the living body, the attachment surface being opposite to a direction in which the first layer member projects; and
a housing space for the sensor body, the housing space being enclosed by the first layer member and the second layer member, in which
a permeating amount of water vapor entering the housing space through the second layer member at a first temperature is smaller than a permeating amount of water vapor exiting from the housing space through the first layer member at a second temperature lower than the first temperature.

<2> The biological sensor according to <1>, in which
the first layer member includes
   a cover member including at least a part of the housing space, and
   a base provided at the cover member toward the second layer member.

<3> The biological sensor according to <2>, further including:
a through-hole that communicates with the housing space of the cover member; and
a moisture permeable film provided at the through-hole.

<4> The biological sensor according to any one of <1> to <3>, in which
the second layer member includes an adhesive layer on a surface of the second layer member opposite to the first layer member.

<5> The biological sensor according to any one of <1> to <4>, further including:
an electrode on the surface of the first layer member toward the second layer member, the electrode being connected to the sensor body.

<6> The biological sensor according any one of <2> to <5>, further including:
the electrode on the surface of the first layer member toward the second layer member, the electrode being connected to the sensor body, in which
the first layer member includes an adhesive layer on a surface of the base toward the second layer member, the adhesive layer being attached to the electrode.

<7> The biological sensor according to <5> or <6>, in which
the second layer member is provided such that the electrode is exposed at a lower surface of the first layer member.

<8> The biological sensor according to any one of <2> to <>>, in which
the base includes a jutting-out portion in at least a part of an outer peripheral portion of the base, the jutting-out portion projecting beyond at least one of an outer peripheral portion of the cover member or an outer peripheral portion of the second layer member.

<9> The biological sensor according to any one of <2> to <8>, in which
the first layer member includes an adhesive layer on a surface of the base opposite to the second layer member.

<10> The biological sensor according to any one of <2> to <9>, in which
the base includes a polyurethane-based thermoplastic elastomer.

The present application claims priority to Japanese Patent Application No. 2023-017080, filed on Feb. 7, 2023 with the Japan Patent Office, and the entire contents of the above application are incorporated herein by reference.

### REFERENCE SIGNS LIST

1 Biological sensor
2 Skin
10 First layer member
11 Cover member
12 Upper sheet
12A, 121A, 122A Jutting-out portion
12a, 111b, 121a, 122a Through-hole
13 Moisture permeable film
20,20A,20B Electrode
30 Sensor portion
31 Flexible substrate
32 Sensor body
33A, 33B Connection portion
34 Battery
40 Second layer member
41 Second base
42 Lower adhesive layer
43 Second adhesive layer
44 Groove
111 Projection
111a Recess
112A, 112B Flat portion
121 First base
122 First adhesive layer
123 Upper adhesive layer

1-11. A biological sensor to be attached to a living body, the biological sensor comprising:
a sensor body configured to obtain biological information;
a first layer member having a projecting shape so as to cover the sensor body;
a second layer member that is attached to a surface of the first layer member and includes an attachment surface to be attached to the living body, the attachment surface being opposite to a direction in which the first layer member projects; and
a housing space for the sensor body, the housing space being enclosed by the first layer member and the second layer member, wherein
a permeating amount of water vapor entering the housing space through the second layer member at a first temperature is smaller than a permeating amount of water vapor exiting from the housing space through the first layer member at a second temperature lower than the first temperature.

2. The biological sensor according to claim 1, wherein
the first layer member includes
a cover member including at least a part of the housing space, and
a base provided at the cover member toward the second layer member.

3. The biological sensor according to claim 2, further comprising:
a through-hole that communicates with the housing space of the cover member; and
a moisture permeable film provided at the through-hole.

4. The biological sensor according to claim 1, wherein
the second layer member includes an adhesive layer on a surface of the second layer member opposite to the first layer member.

5. The biological sensor according to claim 1, further comprising:
an electrode on the surface of the first layer member toward the second layer member, the electrode being connected to the sensor body.

6. The biological sensor according to claim 2, further comprising:
an electrode on the surface of the first layer member toward the second layer member, the electrode being connected to the sensor body, wherein
the first layer member includes an adhesive layer on a surface of the base toward the second layer member, the adhesive layer being attached to the electrode.

7. The biological sensor according to claim 5, wherein
the second layer member is provided such that the electrode is exposed at a lower surface of the first layer member.

8. The biological sensor according to claim 2, wherein
the base includes a jutting-out portion in at least a part of an outer peripheral portion of the base, the jutting-out portion projecting beyond at least one of an outer peripheral portion of the cover member or an outer peripheral portion of the second layer member.

9. The biological sensor according to claim 2, wherein
the first layer member includes an adhesive layer on a surface of the base opposite to the second layer member.

10. The biological sensor according to claim 2, wherein
the base includes a polyurethane-based thermoplastic elastomer.

11. The biological sensor according to claim 6, wherein
the second layer member is provided such that the electrode is exposed at a lower surface of the first layer member.
